(19) United States
(12) Patent Application Publication
(10) Pub. No.: US 2011/0282074 A1
(43) Pub. Date: Nov. 17, 2011

(54) Process for Preparing Substantially Pure Simvastatin

(75) Inventors: Sugata Chatterjee, Vadodara (IN); Ajay Singh Rawat, Vadodara (IN); Neeraj Kumar, Vadodara (IN); Jetti Rajanikanth, Vizag (IN); Kundan Singh Shekhawat, Pin (IN); Jigar H. Shah, Vadodara (IN); P. Venkateswarlu, Prakasam (IN)

(73) Assignee: Sterling Biotech Research Center a Corporate Research Centre of Sterling Biotech Limited, Vadodara (IN)

(21) Appl. No.: 12/832,263

(22) Filed: Jul. 8, 2010

(30) Foreign Application Priority Data
Mar. 15, 2010 (IN) 676/MUM/2010

(51) Int. Cl. C07D 309/30

(52) U.S. Cl. 549/292

(57) ABSTRACT

This invention relates to an improved process for preparing substantially pure simvastatin (I), chemically known as (1S,3R,7S,8S,8aR)-8-[2-[(2R,4R)-4-hydroxy-6-oxotetrahydro-2-H-pyran-2-yl]ethyl]-3 ,7-dimeth-yl-1,2,3,7,8,8a-Hexahydronaphthalen-1-yl2,2-dimethyl butanoate, which comprises of:
a) treating lovastatin (II) with an alkali metal hydroxide in a chosen suitable alcoholic solvent followed by relactonization to obtain the diol lactone intermediate (III) in a single vessel.
b) selective silylation of 4-hydroxy group of diol lactone intermediate (III) with a chosen suitable silylating reagent to obtain mono silylated intermediate diol lactone (IV).
c) acylation of the mono silylated intermediate (IV) to form silylated simvastatin (V)

Or optionally,
preparing silylated simvastatin (V) starting from Lovastatin (II) without isolating diol lactone (III) and monosilylated diol lactone (IV) and
d) finally, removal of the silyl protecting group on silylated simvastatin (V) followed by purification to provide substantially pure simvastatin (I).

The present application claims priority from Indian patent Application No. 676/MUM/2010 filed on Mar. 15, 2010 which is incorporated herein by reference.

### FIELD

The present disclosure relates to an improved, scalable, economical and environmentally benign procedure for preparing substantially pure simvastatin (I) chemically known as (1S,3R,7S,8S,8aR)-8-[2-[(2R,4R)-4-hydroxy-6-oxotetrahydro-2-H-pyran-2-yl]ethyl]-3,7-dimeth-yl-1,2,3,7,8,8a-hexahydronaphth-alen-1-yl 2,2-dimethylbutanoate.

Simvastatin (Zocor, Lipex, Sinvacor, Sivastin) is clinically used as an anti-hypercholesterolemic agent. Simvastatin reduces serum cholesterol levels and slows the progression of atherosclerosis. Simvastatin like lovastatin is a competitive and reversible inhibitor of the enzyme 3-hydroxy-3-methylglutaryl coenzyme A reductase (HMG-CoA reductase), which catalyzes the NADPH dependent reduction of 3-hydroxy-3-methylglutaryl-CoA to mevalonate.

### BACKGROUND

Simvastatin described chemically as (1S,3R,7S,8S,8aR)-8-[2[(2R,4R)-4-hydro-oxy-6-oxotetrahydro-2H-pyran-2-yl]ethyl]-3,7-dimethyl-1,2,3,7,8,8a-hexahydro-napthalen-1-yl 2,2-dimethyl butanoate has the following structural formula I.

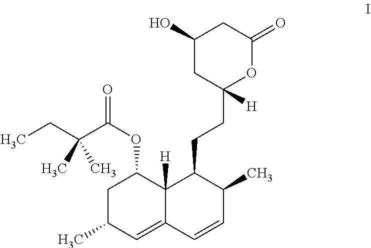

Synthesis of simvastatin from lovastatin (II) was first disclosed in U.S. Pat. No. 4,444,784 by Merck & Co., Inc. In the process disclosed in this patent, lovastatin (II) was hydrolyzed with lithium hydroxide and water to remove 2-methyl-butyryl side chain with concomitant opening of the lactone ring, to produce trihydroxy acid which is eventually heated for relactonisation to obtain diol lactone (III).

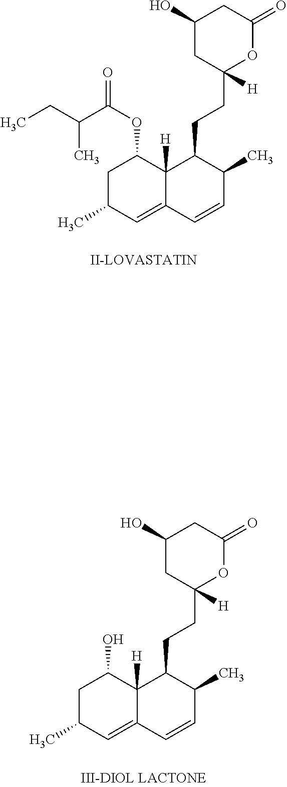

The hydroxy group in the lactone ring is then protected with tertiary butyl dimethyl chlorosilane (TBDMSiCl), imidazole and N,N-dimethylformamide to obtain monosilylated lactone (IV).

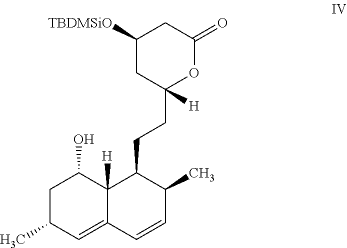

and then hydroxyl group at C-8 position of hexahydronaphthalene ring is acylated with 4-N,N-dimethylaminopyridine (DMAP), pyridine and pivaloyl chloride to produce silylated simvastatin (V).

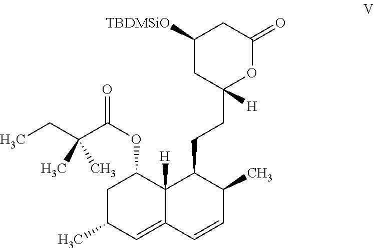

Finally the tertiary butyl dimethyl silyl protecting group is removed using tertra n-butylammonium fluoride (TBAF) with THF and acetic acid to produce title compound (I).

In U.S. Pat. No. 5,159,104, EP 0511867 filed by Merck and U.S. Pat. No. 6,331,641, the reaction starts with treating diol lactone with DMAP and pyridine to form 4-acylated diol lactone, followed by acylation with 2,2-dimethylbutyryl chloride using DMAP instead of 4-pyrrolidino pyridine. Pyridine was used as solvent and acetic anhydride was used in place of tertiary butyldimethylchlorosilane (TBDMSCl) to form 4-acyl simvastatin. Later on methanol and HCl was used to form simvastatin followed by using NaOH and methanol: ammonium hydroxide mixture to form ammonium salt of simvastatin. This was washed with water and glacial acetic acid, butylated hydrorxy anisole was used to form simvastatin. Since pyridine was used as solvent, it suffers from the disadvantage of evaporating and the product needs to be concentrated with ethyl acetate and NaCl or copper sulfate.

U.S. Pat. No. 6,384,238 mainly describes the acylation reaction using metal halides preferably LiCl or LiBr in solvent such as pyridine, collidine, acetonitrile, THF preferably pyridine and 2,2-dimethylbutyryl chloride was added. The reaction requires elevated temperature of 75° C. to 110° C. Lithium chloride or bromides are hazardous and corrosive in nature. So its use in the industry is not very feasible. Moreover, LiBr is hygroscopic, lowers the yield, by-product is produced besides the requirement of a high temperature of 135° C.

In U.S. Pat. No. 6,252,091 the reaction starts with diol lactone which is produced as per process mentioned in EP-B-33-538, EP-B-22-478 and WO 97/05269, silylation and acylation takes place in single pot by adding tertiary-butyldimethyl chlorosilane (TBDMSCl), N-methylimidazole and then adding 2,2-dimethyl butyryl chloride, but over all time required for the reaction is almost 48 hours, the product yield reported is 93%.

In U.S. Pat. No. 6,576,775, triphenylphosphine (TPP), dichloroethane, hexachloroethane, 2,2-dimethylbutyric acid are used for both protection of hydroxyl group as well as esterification. The reaction takes 21 hours. Using TPP has the disadvantage of burdening the effluent with TPPO and work up is tedious.

In U.S. Pat. No. 6,833,461, the main focus is on deprotection of silylated simvastatin, though the entire process for synthesis of simvastatin is mentioned in the description from lovastatin. Lovastatin is hydrolyzed in potassium tertiary butoxide in THF and lactonization of the hydrolyzed compound is done by refluxing in toluene. Silylation is done in conventional method using TBDMS-Cl, pyridine or imidazole in solvent such as DCM and acetonitrile. Acylation done using triphenyl phosphine, 2,2-dimethyl butyric acid and N bromosuccinimide as halogenating agent , later on N,N-dimethylaniline is added instead of DMAP. The focus of their invention was in deprotection of silylated simvastatin to obtain simvastatin, which was done using concentrated HCl in a reaction solvent mainly consisting THF or 1,3 dioxane or 1,4 dioxane. Though the Korean Publication No. 2000-15179 showed few modification wherein t-BuOk is used in hydrolysis and acyloxy triphenylphosphonium salts in acylation, use of t-BuOk is expensive and causes unwanted side reaction and gives low yield and using triphenyl phosphine in the acylation has again the same drawback of complicated purification procedures and effluent burden.

PCT publication WO 2003/057684, discloses a method of preparing simvastatin from lovastatin which is similar to Korean Patent Publication 10-1985-669 and U.S. Pat. No. 4,444,784. Lovastatin is treated with potassium hydroxide dissolved in water and methanol, then relactonizing and protecting the hydroxyl group on the lactone ring in presence of TBDMSCl, DCM and imidazole to obtain monosilylated diol lactone, followed by acylation with 2,2-dimethyl butyryl chloride or bromide in toluene and finally removing the silyl protecting group on the lactone ring to obtain simvastatin. The drawback is when acylation is done with toluene at high temperature for longer time it produces lots of impurities.

In WO/2005/058861 the silylation was done using di-substituted silyl dichloride to produce diol lactone dimer and acylation takes place with 2,2-dimethyl butyryl chloride to produce simvastatin dimer and then deprotection of the simvastatin dimer to produce simvastatin.

PCT publication WO 2007/096753 describes conversion of lovastatin using sodium hydroxide in methanolic solution to triol acid and then using toluene and water to form diol lactone. The diol lactone is silylated with TBDMSCl in presence of imidazole and DMF. In acylation step, cyclohexane is used as solvent; along with DMAP and pyridine as additives. Then 2,2-dimethyl butyryl chloride is used to form silylated simvastatin. This procedure requires additional purification step because of formation of olefin impurities.

### Problems in Methodologies Disclosed in Prior Art

The patent procedures where pyridine is used as reaction solvent, have considerable drawback on the industrial scale. Besides its toxic effects, products coming out of pyridine require thorough purification by multiple washes with acidified water thereby reducing the production yield and increasing the cost of the corresponding ester. Additionally large amount of unreacted starting material along with undesired by-products formed during the process complicates recovery of the final product.

### Solution to Minimize the Problems

The present disclosure utilizes n-heptane as solvent which has the following advantages:
1. N-heptane is considered as an environmentally benign solvent.
2. Toxicity data available from OSHA PEL, ACGIH TLV, DFG MAK (Exposure Controls) represents n-heptane as safer solvent compared to cyclohexane.
3. The method is reproducible, easily scalable and n-heptane can be almost quantitatively recovered for reuse
4. Extraction by n-heptane is more efficient than that by cyclohexane.
The present invention thus addresses all the needs for an industrially feasible, scalable, commercially viable and eco-friendly process for the preparation of simvastatin.

### SUMMARY

The objective of the present disclosure is to overcome the observed drawbacks in the prior art and provide an improved, scalable, economical and environmentally benign procedure for preparing substantially pure simvastatin (I).

Another objective of the present disclosure is to provide an improved, scalable, economical and environmentally benign procedure for preparing substantially pure simvastatin (I), wherein lovastatin (II) is first converted to dial lactone intermediate (III) without isolating the intermediate triol acid in a single step avoiding the cumbersome process of isolating triol acid.

Another objective of the present disclosure is to provide an improved, scalable, economical and environmentally benign procedure for preparing substantially pure simvastatin (I), by chemoselective silylation of the less hindered 01-1 group furnishing mono silylated diol lactone intermediate (IV) followed by its acylation to form silylated simvastatin (V), the key intermediate used in the preparation of the title compound of formula (I), is performed in a suitable eco-friendly and less toxic organic solvent.

Another important objective of the present disclosure is to overcome the olefin impurity (ies) encountered in the prior art during the acylation stage and provide an improved, scalable, economical and environmentally benign procedure for preparing substantially pure simvastatin (I).

Another significant objective of the present disclosure is to provide an improved, scalable, economical and environmentally benign procedure for preparing substantially pure simvastatin (I), wherein desilylation of silylated simvastatin (V) directly yields simvastatin (I) in desired quality and yield.

These and other objectives as mentioned will be apparent in the following detailed description.

### DETAILED DESCRIPTION

The process of the present disclosure, illustrated in scheme I, is described below.

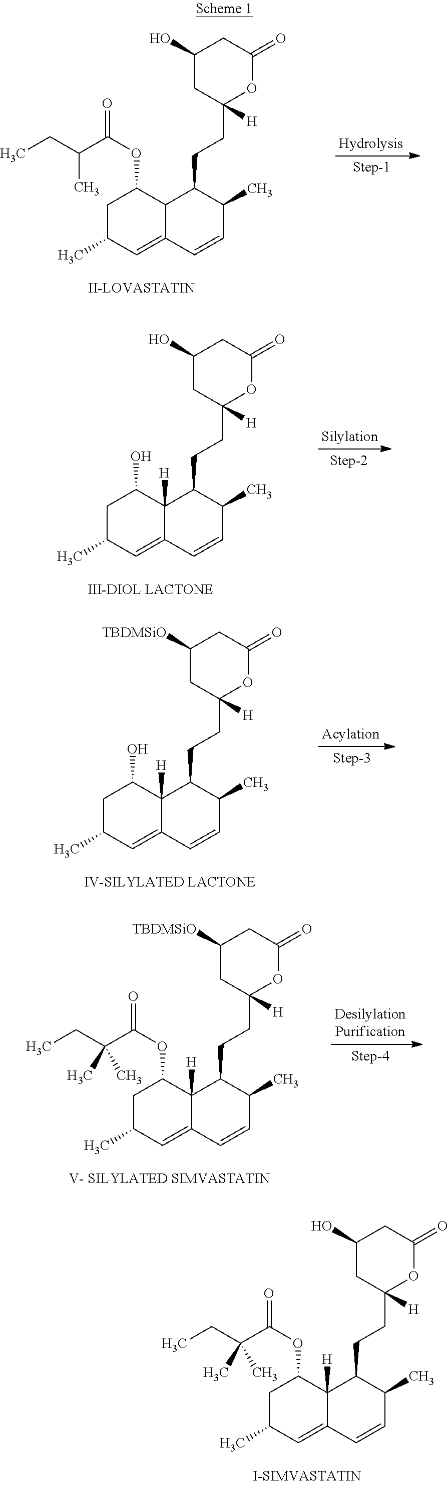

Accordingly, the present disclosure provides an improved, scalable, economical and environmentally benign procedure for preparing substantially pure simvastatin (I) which comprises of,

a) treating lovastatin (H) with an alkali metal hydroxide in a chosen suitable alcoholic solvent followed by relactonization to give the diol lactone intermediate (III) in single step.

b) selective silylation of the 4-hydroxy group of the thus obtained diol lactone intermediate (III) with a chosen suitable silylating reagent to give mono silylated intermediate (IV).

c) acylation of the so obtained mono silylated intermediate (IV) to form silylated simvastatin (V) Or optionally preparing silylated Simvastatin (V) in a single pot using a single solvent starting from Lovastatin (II) without isolating diol lactone intermediate (III) and monosilylated intermediate (IV) &

d) Finally, removal of the hydroxyl protecting group on silylated simvastatin (V) followed by purification to provide substantially pure simvastatin (I).

The present disclosure provides a process wherein conversion of lovastatin (II) to diol lactone intermediate (III) does not involve the isolation of triol acid.

The present disclosure further provides a process wherein the suitable alkali and alkaline earth metal hydroxide used for hydrolysis of lovastatin (II) is chosen from lithium hydroxide, potassium hydroxide, sodium hydroxide, magnesium hydroxide, calcium hydroxide etc., most preferably sodium hydroxide.

The present disclosure further provides a process wherein 1.0 to 15.0 molar equivalents of the chosen suitable alkali metal hydroxide is used.

The present disclosure further provides a process wherein the suitable alcoholic solvent used for the hydrolysis of lovastatin (II) is chosen from tertiary butanol, n-butanol, 2-propanol, 1-propanol, ethanol and methanol etc., most preferably methanol.

The present disclosure further provides a process wherein the chosen suitable alcoholic solvent for the hydrolysis of lovastatin (II) is used as such or in combination with water.

The present disclosure furthers provides a process wherein the hydrolysis of lovastatin (II) is carried out at a temperature ranging from ambient to reflux temperature of the chosen reaction medium.

The present disclosure furthers provides a process wherein the in-situ relactonization of the triol acid obtained after the hydrolysis of lovastatin (II) to furnish diol lactone intermediate (HI) is carried out optionally in a suitable single halogenated or non halogenated organic solvent or in a binary mixture of one or more halogenated or non halogenated organic solvent or both.

The present disclosure furthers provides a process wherein the suitable halogenated solvent used for the in situ relactonization of the triol acid obtained after the hydrolysis of lovastatin (II) to furnish dial lactone intermediate (III) is chosen from dichloroethane, carbontetrachloride, chloroform and dichloromethane etc., most preferably dichloromethane.

The present disclosure furthers provides a process wherein the suitable non halogenated solvent used for in-situ relactonization of the triol acid obtained after the hydrolysis of lovastatin (II) to furnish diol lactone intermediate (HI) is chosen from a suitable aprotic solvent such as for example dimethylformamide, dimethylsulphoxide, tetrahydrofuran, dioxane, diethylether, dimethoxymethane, toluene, xylene, hexane, heptane etc., most preferably toluene. The in-situ relactonization of the triol acid obtained after the hydrolysis of lovastatin (II) to furnish diol lactone intermediate (III) may also be preferably carried out in a protic solvent such as for example tertiary butanol, n-butanol, 2-propanol, 1-propanol, ethanol and methanol etc., most preferably in methanol.

The present disclosure furthers provides a process wherein the in situ relactonisation of the triol acid obtained after the hydrolysis of lovastatin (II) to furnish diol lactone intermediate (III) is carried out at a temperature ranging from ambient to reflux temperature of the chosen reaction medium.

The present disclosure further provides a process wherein the selective silylation of 4-hydroxy group of diol lactone intermediate (III) is carried out with a suitable silylating agent chosen from tertiarybutyldimethylsilyl chloride, triethylsilyl chloride, trimethylsilyl chloride etc., and most preferably tertiary butyldimethylsilyl chloride.

The present disclosure further provides a process wherein the selective silylation of 4-hydroxy group of diol lactone intermediate (III) is carried out in the presence of a suitable organic base such as for example triethylamine, diisopropylethylamine, pyridine, piperidine, pyrrolidine, dimethylaminopyridine, imidazole etc., most preferably imidazole.

The present disclosure further provides a process wherein the selective silylation of 4-hydroxy group of diol lactone intermediate (III) is carried out in a suitable aprotic solvent chosen from dimethylformamide, dimethylsulphoxide, toluene, dichloromethane, tetrahydrofuran, dioxane etc., most preferably dimethylformamide.

The present disclosure further provides a process wherein the suitable reaction temperature for the selective silylation of 4-hydroxy group of diol lactone intermediate (III) when carried out in-situ ranges from room temperature to reflux temperature of the chosen solvent.

The present disclosure further provides a process wherein silylated dial lactone (IV) is acylated with 2,2-dimethyl butyryl chloride in the presence of a suitable organic base such as for example triethylamine, diisopropylethylamine, pyridine, piperidine, pyrrolidine, dimethylaminopyridine, imidazole etc., most preferably pyridine.

The present disclosure further provides a process wherein the acylation of silylated diol lactone (IV) to give silylated simvastatin (V) is performed in a less toxic, non-hazardous and recyclable organic solvent.

The present disclosure further provides a process wherein the less toxic, non-hazardous and recyclable organic solvent used in the acylation of silylated diol lactone (IV) to give silylated simvastatin (V) is chosen from a suitable acyclic C-5 to C-10 linear or branched hydrocarbon.

The present disclosure further provides a process wherein the suitable less toxic, non-hazardous and recyclable acyclic C-5 to C-10 linear or branched hydrocarbon solvent used in the acylation of silylated diol lactone (IV) to give silylated simvastatin (V) is chosen from octane, n-heptane, heptanes, n-hexane, hexanes, pentane etc., most preferably n-heptane.

The present disclosure further provides a process wherein silylated simvastatin (V) is prepared without isolating diol lactone intermediate (III) and monosilylated intermediate (IV).

The present disclosure further provides a process wherein desilylation of silylated simvastatin (V) followed by purification provides substantially pure simvastatin (I).

The present disclosure further provides a process wherein the crude simvastatin obtained after desilylation is purified in one step by way of treating with silica gel to provide substantially pure simvastatin (I).

The present disclosure further provides a process wherein the crude simvastatin obtained after desilylation is purified optionally by filtration over silica gel.

The preferable range for the mesh size of silica gel used for the purification of crude simvastatin to provide substantially pure simvastatin (I) lies between 40-400, preferably 100-200.

The preferable amount of silica gel used for filtration lies anywhere between 0.5 times to 50.0 times w/w.

The present invention further provides a process wherein the suitable organic solvent used to purify crude simvastatin by filtration over silica gel is a single or mixture of solvents chosen from ethylacetate, butylacetate, diisopropyl ether, diethylether, tertiarybutylmethylether, n-hexane, hexanes, cyclohexane, heptane, methanol, ethanol, 2-propanol, 1-propanol, t-butanol, s-butanol etc, most preferably a mixture of ethylacetate and hexane.

The following examples illustrate, but in no way limit the scope of the new process described in this disclosure. Any deviation from this, apparent and obvious to a person skilled in the art of organic synthesis, forms part of this invention, though not explicitly substantiated.

### EXAMPLES

### Example 1

### Preparation of 6(R)-[2-(8′(S)-hydroxy-2′(S),6′(R)-1′,2′,6′,7′,8′,8a′(R)-hexahydronapthyl-1′(S)-ethyl]-4-(R)-hydroxy-3,4,5,6-tetrahydro-2H-pyran-2-on (Diol Lactone)

NaOH (100.0 gm) was charged followed by methanol (500.0 ml) at 22° C.-25° C. Temperature rose to 55° C.-60° C. When temperature was came down to 40° C.-45° C., lovastatin (100.0 gm) was charged followed by adding methanol (200.0 ml). Temperature was raised to reflux (oil bath temp 80° C.). The reaction mass was stirred at reflux temperature till completion was observed by HPLC. The reaction was completed in 35 hours. The reaction was then cooled to 23° C.-25° C. and 150.0 ml of demineralized (DM) water was added followed by drop wise addition of conc. hydrochloric acid to adjust the pH to 7.5-8.0. Temperature rose from 24° C.-32° C. and care was taken that it does not exceed 35° C. Methanol and water was distilled out completely under vacuum at 60° C. The residue was taken in DM water (300.0 ml) and dichloromethane (150.0 ml) and pH was adjusted to 1.5-2.0 with conc. hydrochloric acid at 5° C.-10° C. The mixture was stirred for 1 hr at 5° C.-10° C. Water and dichloromethane were again distilled out completely and to the remaining residue toluene (100.0 ml) was added and then distilled completely. To this residue 4.0 liter DM water was added and the mixture stirred for 1 hr at 10° C.-15° C. The solid was filtered, washed with DM water (1000.0 ml) and dried under high vacuum at 60° C. for 24 hrs. This dried material was taken in 500.0 ml hexane and stirred for 1 hr at 15° C.-20° C. and the solid was filtered. This solid material was dried at 45° C.-50° C.

### Example 2

### Preparation of 6(R)-[2-(8′(S)-hydroxy-2′(S),6′(R)-dimethyl-1′,2′,6′,7′,8′,8a′(R)-hexahydronapthyl-1′(S)-ethyl]-4-(R)-dimethyl-tert-butylsilyl-1-oxy)-3,4,5,6-tetrahydro-2H-pyran-2-one (Monosilylated Diol Lactone)

Imidazole (66.4 gm) was dissolved in DMF (150 ml) under nitrogen at 25° C.-30° C. The clear solution obtained was cooled to 15° C.-20° C. followed by drop wise addition of TBDMSCl (73.2 gm in 200 ml DMF). To this solution diol lactone (78.0 gm) was added at 15° C.-20° C. followed by DMF (120.0 ml). The reaction mass was stirred for 3-5 hrs at 15° C.-20° C. The reaction progress was monitored by HPLC. After completion of reaction, 160.0 ml of water was added to the reaction mass and stirred for 1 hour at 20° C.-25° C. The precipitated solid was filtered, the cake was triturated with water (400 ml) and the solvents removed by applying high vacuum. The compound was dried at 50° C. under vacuum till a constant weight was attained.

### Example 3

### Preparation of 6(R)-[2-(8′(S)-2″.2″-dimethylbutyrloxy-2′(S),6′(R)-dimethyl-1′,2′,6′,7′,8′,8a′(R)-hexahydronapthyl-1′(S)-ethyl]-4-(R)-dimethyl-tert-butylsilyloxy)-3,4,5,6-tetrahydro-2H-pyran-2-one (Silylated simvastatin)

Silylated lactone (100 g, 0.23 mole), n-heptane (1.3 lts), and dimethylamino pyridine (DMAP) (9.86 g, 0.08 mole) and pyridine (145.93 g, 1.84 mole) were added under stirring at 20° C.-25° C. under nitrogen atmosphere. The content was stirred for 15 minutes at 20° C.-25° C. 2,2-Dimethylbutyryl chloride (102.4 g, 0.76 moles) was added followed by flushing with n-heptane (200 ml) at 20° C.-25° C., the temperature slowly raised to reflux. The reaction mass refluxed for 36 hours under nitrogen atmosphere. The progress of reaction was monitored by TLC, and when the reaction was completed, the reaction mass was cooled to 20° C.-25° C. and added to (1.0 lts) water, stirred for 30 minutes and settled for another 30 minutes at 20° C.-25° C., the lower aqueous layer separated, followed by the washing of organic phase with 1 lit of 0.2 N HCl, 1 lit, 10% aqueous solution of NaHCO3. Finally the organic layer was washed with 1 liter water. The organic layer (n-heptane) was removed under vacuum completely to furnish an oil (122.0 g).

### Example 4

### Preparation of 6(R)-[2-(8′(S)-2″.2″-dimethylbutyrloxy-2′(S),6′(R)-dimethyl-1′,2′,6′,7′,8′,8a′(R)-hexahydronapthyl-1′(S)-ethyl]-4-(R)-dimethyl-tert-butylsilyloxy)-3,4,5,6-tetrahydro-2H-pyran-2-one (Silylated simvastatin)—without isolating diol lactone (III) and monosilylated dial lactone (IV).

Lovastatin (50 g, 0.123 moles) and sodium hydroxide (50 g, 1.25 moles) were taken in methanol (350 ml) and heated to reflux for 24 hrs. Then the reaction mass was concentrated completely which was followed by addition of water. The pH of the reaction mixture was then adjusted below 2.0 using concentrated hydrochloric acid and stirred for half an hour at ambient temperature. The precipitated solid (triol acid) was then filtered and washed with cold water. The wet triol acid was then taken in toluene (600 ml) and water was removed azeotropically. Then the reaction mixture was cooled to 25-30° C. after which imidazole (31.0 g, 0.455 moles) followed by tert-butyldimethylsilylchloride (34.2 g, 0.227 moles) was added. The obtained reaction mixture was maintained at ˜85° C. for 2 hours and then cooled to room temperature. The reaction mixture was then washed with 0.2N aqueous hydrochloric acid solution, 10% sodium bicarbonate solution, water and then dried over Sodium Sulfate. To this dried toluene layer Dimethyl amino pyridine (DMAP) (1.4 g, 0.011 moles) and Imidazole (18.5 g, 0.271 moles) were added at 20° C. to 25° C. under nitrogen atmosphere. The contents were stirred for 15 minutes at 20° C. to 25° C. after which 2,2-dimethylbutyryl chloride (36.7 g, 0.272 moles) was added at 20° C. to 25° C. Then the temperature of the reaction mixture was raised to reflux and was maintained at reflux for 24 hours. Then the reaction mixture was brought to 20° C. to 25° C. and washed with 0.2N aqueous hydrochloric acid solution. The separated organic layer was then dried over sodium sulfate and concentrated completely under vacuum to give a brown oil. (Weight 64.0 g).

### Example 5

### Preparation of 6(R)-[2-(8′(S)-2″,2″-dimethylbutyryloxy-2′(S), 6′(R)-dimethyl-1′,2′,6′,7′,8′,8a′(R)-hexahydronapthyl-1′(S)-ethyl]-4-(R)-hydroxy-3,4,5,6-tetrahydro-2H-pyran-2-one (Simvastatin). Using TBAF:

Silylated simvastatin (25 gm) and THF (360 ml) were added under stirring in a round bottom flask at 25° C.-35° C., the mixture stirred for 15 minutes and acetic acid (21 gm) was added. The reaction mass was cooled to 15° C.-20° C. A solution of tetrabutyl ammonium fluoride in THF was added into the reaction mass, stirred for 30-35 hours at 18° C.-22° C. and the reaction mass was poured in water. DCM was added, pH adjusted to neutral with sodium bicarbonate solution, stirred for 30 minutes and settled for 30 minutes. The aqueous layer was separated and water was added to organic layer. The organic layer was separated, charcoal was added, stirred for 60 minutes and filtered through hyflosupercel bed. The filtrate was concentrated and the crude obtained was subjected to silica treatment using 15% ethyl acetate-hexane mixture furnishing 12.5 g of simvastatin (purity by HPLC 99.5%). No further purification was required.

### Example 6

### Preparation of 6(R)-[2-(8′(S)-2″,2″-dimethylbutyryloxy-2′(S), 6′(R)-dimethyl-1′,2′,6′,7′,8′,8a′(R)-hexahydronapthyl-1′(S)-ethyl]-4-(R)-hydroxy-3,4,5,6-tetrahydro-2H-pyran-2-one (Simvastatin). Using aqueous HF

Silylated simvastatin (56.0 gm) was dissolved in 560 ml of acetonitrile at 22-25° C. and then further cooled to 0-5° C. Then 48% aqueous hydrofluoric acid (110.8 ml) diluted in acetonitrile (840 ml) was added in 30 minutes at the same temperature. Then the temperature was raised to 22-25° C. and the reaction was maintained at this temperature for 2.0 hours. Then the pH of the reaction mixture was adjusted to ˜7.0 by the addition of 10% sodium bicarbonate solution. The reaction mixture was stirred for 15.0 minutes at 22-25° C. and the layer was separated. The aqueous layer was washed twice with ethyl acetate (560 ml×2.0). The total organic layer was then combined concentrated completely under vacuum below 40° C. to yield a thick oily residue. The Last printed Jun. 14, 2010 9:55:00 AMPage 15 of 21 thick oily residue thus obtained was dissolved in dichloromethane (560 ml) and was washed twice with water (140.0 ml). The organic layer was separated, charcoal was added, stirred for 60 minutes and filtered through hyflosupercel bed. The filtrate was concentrated and the crude obtained was subjected to silica treatment using 15% ethyl acetate-hexane mixture furnishing 25.0 g of simvastatin (purity by HPLC ˜99.5%).

### HPLC Method Used for Simvastatin Analysis

Mobile Phase A: Acetonitrile: diluted phosphoric acid (50:50) [Preparation method for diluted phosphoric acid solution:—Transfer 1 ml of phosphoric acid to a 1-L volumetric flask, and dilute with water to volume].

Mobile phase B: Transfer 1 ml of phosphoric acid to a 1-L volumetric flask, and dilute with acetonitrile to volume.

Diluent: Acetonitrile: Buffer solution (3:2) [Preparation method for buffer solution:—Prepare a solution containing 1.4 g of monobasic potassium phosphate per litre and adjust with Phosphoric acid to a pH of 4.0].

### Column: Peerless C18 33×4.6 mm, 3 u

### Detector: UV at 238 nm

Injection volume: 5 μl
Run time: 13 min
Column temp: 25° C.

### Gradient:

| Time (min) | Flow rate (ml/min) | Mobile phase A | Mobile phase B |
|---|---|---|---|
| 0 | 3.0 | 100 | 0 |
| 4.5 | 3.0 | 100 | 0 |
| 4.6 | 3.0 | 95 | 5 |
| 8 | 3.0 | 25 | 75 |
| 11.5 | 3.0 | 25 | 75 |
| 11.6 | 3.0 | 100 | 0 |
| 13 | 3.0 | 100 | 0 |

Isolated sample of final simvastatin: Concentration 1.5 mg/ml
Retention Time Simvastatin: ˜3.4 Minutes.

We claim:

1. An improved, scalable, economical and environment friendly procedure for preparing substantially pure simvastatin (1), chemically known as (1S,3R, 7S,8S,8aR)-8-[2-[(2R,4R)-4-hydroxy-6-oxotetrahydro-2-H-pyran-2-yl]ethyl]-3,7-dimeth-yl-1,2,3,7,8,8a-Hexahydro naphthalen-1-yl2,2-dimethylbutanoate, 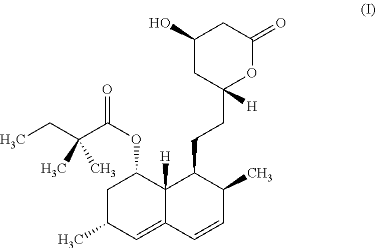
which comprises of
a) treating lovastatin (II) with an alkali metal hydroxide in a chosen suitable alcoholic solvent followed by relactonization to obtain the diol lactone intermediate (III) in a single vessel.
b) selective silylation of 4-hydroxy group of diol lactone intermediate (III) with a chosen suitable silylating reagent to obtain mono silylated intermediate diol lactone (IV).
c) acylation of the mono silylated intermediate (IV) to form silylated simvastatin (V)
Or optionally,
preparing silylated simvastatin (V) starting from Lovastatin (II) without isolating diol lactone (III) and monosilylated diol lactone (IV) and
d) finally, removal of the silyl protecting group on silylated simvastatin (V) followed by purification to provide substantially pure simvastatin (I).

2. The process as claimed in claim 1, wherein hydrolysis of lovastatin (II) to obtain diol lactone intermediate (III) does not involve isolation of triol acid.

3. The process as claimed in claim 1, wherein monosilylated intermediate (IV) is acylated with 2,2-dimethyl butyryl chloride in the presence of an organic base such as for example triethylamine, diisopropylethylamine, pyridine, piperidine, pyrrolidine, dimethylaminopyridine, imidazole, most preferably pyridine to obtain silylated simvastatin (V)

4. The process as claimed in claim 3, wherein 1.0 to 8.0 molar equivalents of pyridine is used.

5. The process as claimed in claim 1, wherein acylation of monosilylated intermediate (IV) is carried out in non-hazardous and recyclable acyclic C-5 to C-10 linear or branched hydrocarbon solvent used in the acylation of silylated diol lactone (IV) to give silylated simvastatin (V) is chosen from octane, n-heptane, heptanes, n-hexane, hexanes, pentane etc., most preferably n-heptane.

6. The process as claimed in claim 5, wherein 0.5 to 4.0 molar equivalents of 2,2% dimethylbutyryl chloride is used for acylation.

7. The process as claimed in claim 6, wherein 1.0 to 8.0 molar equivalents of pyridine is used.

8. The process as claimed in claim 5, wherein 0.01 to 1.0 molar equivalents of dimethylamino pyridine is used.

9. The process as claimed in claim 5, wherein silylated simvastatin (V) is prepared without the isolation of diol lactone (III) and monosilylated intermediate (IV) in a single chosen organic solvent.

10. The process as claimed in claim 1, wherein the desilylated simvastatin was purified in one step by way of silica gel treatment to provide substantially pure simvastatin (I).

11. The process as claimed in claim 10, wherein simvastatin obtained after desilylation is purified in one step by way of silica gel batch treatment or silica gel filtration column to provide substantially pure simvastatin (I).

12. The process as claimed in claim 11, wherein the suitable organic solvent used to purify crude simvastatin by silica gel batch treatment or silicagel filtration column is a single or mixture of solvents chosen from ethylacetate, butylacetate, diisopropylether, diethylether, tertiarybutylmethyl ether, n-hexane, hexanes, cyclohexane, heptane, methanol, ethanol, 2-propanol, 1-propanol, t-butanol, sec-butanol etc, most preferably ethyl acetate-hexane mixture

13. The process as claimed in claim 12, wherein the preferable range for the mesh size of silica gel used for the purification of crude simvastatin to provide substantially pure simvastatin (I) lies between 40-400, most preferably 100-200.

14. The process as claimed in claim 12 wherein the preferable amount of silica gel used for batch treatment or silica gel filtration column lies anywhere between 0.5 times to 50.0 times w/w.
